(19) United States
(12) Patent Application Publication
(10) Pub. No.: US 2026/0084090 A1
(43) Pub. Date: Mar. 26, 2026

(54) SYSTEMS AND METHODS FOR A TEMPERATURE SWING ADSORPTION ADSORBENT BED

(71) Applicant: Archaea Energy Inc., Houston, TX (US)

(72) Inventors: Kevin Michael Weatherford, Angel Fire, NM (US); Stephen Jeffery Moore, Rosemount, MN (US); Charles L. Anderson, Wilmington, DE (US); Michael John Mitariten, Pittstown, NJ (US)

(73) Assignee: Archaea Energy Inc., Houston, TX (US)

(21) Appl. No.: 19/334,479

(22) Filed: Sep. 19, 2025

Related U.S. Application Data
(60) Provisional application No. 63/697,226, filed on Sep. 20, 2024.

(51) Int. Cl. B01D 53/04; B01D 53/22; B01D 53/26; C07C 7/144

(52) U.S. Cl. CPC B01D 53/0462; B01D 53/229; B01D 53/261; C07C 7/144; B01D 2253/104; B01D 2253/116; B01D 2256/245; B01D 2257/504; B01D 2257/702; B01D 2257/708; B01D 2257/80; B01D 2258/05

(57) ABSTRACT

In some embodiments, a system, and at least one method relating thereto, comprises a temperature swing adsorption (TSA) vessel comprising: an inlet and an outlet configured to provide a fluid flow through the TSA vessel; and a TSA media disposed within the TSA vessel, wherein the TSA media comprises: a first layer comprising a first material; a second layer comprising a second material configured to remove water from the fluid flow, wherein the second layer is downstream of the first layer with respect to the fluid flow; and a third layer comprising a third material configured to remove one or more hydrocarbons, volatile organic compounds (VOCs), or a combination thereof, from the fluid flow, wherein the third layer is downstream of the second layer with respect to the fluid flow.

### CROSS-REFERENCE TO RELATED APPLICATIONS

This application claims benefit of U.S. provisional patent application Ser. No. 63/697,226 filed Sep. 20, 2024, and entitled “Systems and Methods for a Temperature Swing Adsorption Adsorbent Bed,” which is hereby incorporated herein by reference in its entirety for all purposes.

### STATEMENT REGARDING FEDERALLY SPONSORED RESEARCH OR DEVELOPMENT

Not applicable.

### TECHNICAL FIELD

The disclosure relates generally to the treatment of landfill gas. More particularly, the disclosure relates to systems and methods for removing undesirable contaminants in a stream of landfill gas.

### BACKGROUND

Decomposition and/or anaerobic fermentation of organic materials in a landfill generates biogas. In general, biogas consists primarily of methane and carbon dioxide (CO₂), followed by small quantities of water vapor, nitrogen and oxygen, hydrogen sulfide, non-methane hydrocarbons, inorganic compounds, volatile organic compounds (VOCs), heavy hydrocarbons (e.g., C₆_{⁺}), etc. Certain components of biogas (e.g., methane) are considered to be an attractive renewable energy source, as biogas can be purified and subsequently utilized in downstream application such as thermal applications, generation of electricity, vehicle fuel, or bio-product feedstock. However, current purification techniques are insufficient at removing contaminants (e.g., non-methane hydrocarbons, inorganic compounds, volatile organic compounds (VOCs), heavy hydrocarbons (e.g., C₆_{⁺})) during treatment of biogas. Accordingly, a need exists for systems that removes contaminants to low levels to generate high quality renewable natural gas products such as methane.

### SUMMARY

In some embodiments, a system comprises a temperature swing adsorption (TSA) vessel comprising: an inlet and an outlet configured to provide a fluid flow through the TSA vessel; and a TSA media disposed within the TSA vessel, wherein the TSA media comprises: a first layer comprising a first material; a second layer comprising a second material configured to remove water from the fluid flow, wherein the second layer is downstream of the first layer with respect to the fluid flow; and a third layer comprising a third material configured to remove one or more hydrocarbons, volatile organic compounds (VOCs), or a combination thereof, from the fluid flow, wherein the third layer is downstream of the second layer with respect to the fluid flow, wherein the TSA media is configured to generate a treated gas stream based on the fluid flow, and wherein a volume of the third layer is greater than a volume of the second layer.

In some embodiments, a method comprises providing a compressed gas stream flow to a TSA vessel; contacting the compressed gas stream flow with a first layer of first material within the TSA vessel to generate a first water depleted stream; contacting the first water depleted stream with a second layer of a second material within the TSA vessel to generate a second water depleted stream; and contacting the second water depleted stream with a third layer of a third material within the TSA vessel to generate a treated gas stream.

In some embodiments, a method comprises providing a first material within a first portion of a vessel; providing a second material within a second portion of the vessel; and providing a third material within a third portion of the vessel, thereby generating TSA media within the vessel, and wherein a volume of the third portion is greater than a volume of the second portion.

In some embodiments, a system comprises a temperature swing adsorption (TSA) vessel comprising an inlet and an outlet configured to provide a fluid flow through the TSA vessel; and a TSA media disposed within the TSA vessel, wherein the TSA media comprises: a first layer comprising a first material configured to remove water from the fluid flow, wherein the first layer is downstream with respect to the fluid flow; and a second layer comprising a second material configured to remove one or more hydrocarbons, volatile organic compounds (VOCs), or a combination thereof, from the fluid flow, wherein the second layer is downstream of the first layer with respect to the fluid flow, wherein the TSA media is configured to generate a treated gas stream based on the fluid flow, and wherein a volume of the second layer is greater than a volume of the first layer.

Embodiments described herein comprise a combination of features and characteristics intended to address various shortcomings associated with certain prior devices, systems, and methods. The foregoing has outlined rather broadly the features and technical characteristics of the disclosed embodiments in order that the detailed description that follows may be better understood. The various characteristics and features described above, as well as others, will be readily apparent to those skilled in the art upon reading the following detailed description, and by referring to the accompanying drawings. It should be appreciated that the conception and the specific embodiments disclosed may be readily utilized as a basis for modifying or designing other structures for carrying out the same purposes as the disclosed embodiments. It should also be realized that such equivalent constructions do not depart from the spirit and scope of the principles disclosed herein.

### BRIEF DESCRIPTION OF THE DRAWINGS

For a detailed description of various exemplary embodiments, reference will now be made to the accompanying drawings in which:

FIG. 1 is a schematic diagram of an embodiment of a fluid purification system, in accordance with an aspect of the present disclosure;

FIG. 2 is a flow diagram of a process related to an embodiment of the fluid purification system of FIG. 1, in accordance with an aspect of the present disclosure;

FIG. 3 is a schematic diagram of an embodiment of the biogas purification system of FIG. 1, in accordance with an aspect of the present disclosure;

FIG. 4 is a schematic diagram of an adsorbent bed of a temperature swing adsorption (TSA) system of FIGS. 1 and 3, in accordance with an aspect of the present disclosure;

FIG. 5 is a flow diagram related to the embodiment of the TSA adsorbent bed of FIG. 4, in accordance with an aspect of the present disclosure; and

FIG. 6 is a flow diagram related to the embodiment of the TSA adsorbent bed of FIG. 4, in accordance with an aspect of the present disclosure.

### DETAILED DESCRIPTION

The following discussion is directed to various exemplary embodiments. However, one skilled in the art will understand that the examples disclosed herein have broad application, and that the discussion of any embodiment is meant only to be exemplary of that embodiment, and not intended to suggest that the scope of the disclosure, including the claims, is limited to that embodiment.

Certain terms are used throughout the following description and claims to refer to particular features or components. As one skilled in the art will appreciate, different persons may refer to the same feature or component by different names. This document does not intend to distinguish between components or features that differ in name but not function. The drawing figures are not necessarily to scale. Certain features and components herein may be shown exaggerated in scale or in somewhat schematic form and some details of conventional elements may not be shown in interest of clarity and conciseness.

In the following discussion and in the claims, the terms “including” and “comprising” are used in an open-ended fashion, and thus should be interpreted to mean “including, but not limited to . . . .” As used herein, the phrases “consist(s) of” and “consisting of” are used to refer to exclusive components of a composition, meaning only those expressly recited components are included in the composition; whereas the phrases “consist(s) essentially of” and “consisting essentially of” are used to refer to the primary components of a composition, meaning that only small or trace amounts of components other than the expressly recited components (e.g., impurities, byproducts, etc.) may be included in the composition. For example, a composition consisting of X and Y refers to a composition that only includes X and Y, and thus, does not include any other components; and a composition consisting essentially of X and Y refers to a composition that primarily comprises X and Y, but may include small or trace amounts of components other than X and Y. In embodiments described herein, any such small or trace amounts of components other than those expressly recited following the phrase “consist(s) essentially of” or “consisting essentially of” preferably represent less than 5.0 wt % of the composition, more preferably less than 4.0 wt % of the composition, even more preferably less than 3.0 wt % of the composition, and still more preferably less than 1.0 wt % of the composition. Use of broader terms such as comprises, includes, having, etc. should be understood to provide support for narrower terms such as consisting of, consisting essentially of, comprised substantially of, etc. Use of the term “optionally” with respect to any element of a claim is intended to mean that the subject element is required, or alternatively, is not required. Both alternatives are intended to be within the scope of the claim. When introducing elements of various embodiments of the present disclosure, the articles “a,” “an,” and “the” are intended to mean that there are one or more of the elements.

The term “couple” or “couples” is intended to mean either an indirect or direct connection. Thus, if a first device couples to a second device, that connection may be through a direct engagement between the two devices, or through an indirect connection that is established via other devices, components, nodes, and connections. As used herein, the terms “approximately,” “about,” “substantially,” and the like mean are intended to convey that the value being described may be within a relatively small range of the property value, as those of ordinary skill would understand. In particular, the terms “approximately,” “about,” “substantially,” and the like mean within 10% (i.e., plus or minus 10%) of the recited value, within 5% (i.e., plus or minus 5%) of the recited value, or within 2% (i.e., plus or minus 2%) of the recited value.

Unless the context dictates the contrary, all ranges set forth herein should be interpreted as being inclusive of their endpoints, and open-ended ranges should be interpreted to include only commercially practical values. In addition, with respect to all ranges disclosed herein, such ranges are intended to include any combination of the mentioned upper and lower limits even if the particular combination is not specifically listed. All lists of values should be considered as inclusive of intermediate values unless the context indicates the contrary. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.).

The term “TSA system” refers to one or more adsorbent beds configured to remove contaminants from landfill gas by selectively adsorbing contaminants at specific temperatures. It should be noted that “TSA system,” “TSA unit,” “adsorbent bed,” “media bed,” “TSA media vessel”, and “vessel” may be used interchangeably.

The term “membrane separation stage” refers to one or more semi-permeable membranes configured to remove undesirable components from desirable components of a fluid stream by permitting the desirable components to pass through the one or more semi-permeable membranes based on molecular properties of the desirable components. Desirable components do not pass through the semi-permeable membranes and may be directed to one or more subsequent purification stages.

As used herein, the phrase “based on” as related to a component generating a first fluid “based on” a second fluid, refers to the component generating the first fluid from the second fluid, but not solely from the second fluid. For example, the component may generate the first fluid using the second fluid and one or more additional streams (e.g., recycle streams). As another non-limiting example, an additional component may generate a third fluid from the second fluid, and the component may receive the third fluid from the additional component. In such an example, the component generates the first fluid based on the second fluid and/or the third fluid.

As used herein, a component that is “positioned along”, “positioned upstream”, and “positioned downstream” with respect to a flow path, indicates that the component receives a fluid along the flow path and/or provides, generate, or otherwise modifies a fluid along the flow path.

As used herein, a “flow path” refers to a conduit or route of one or more fluids. A flow path may be formed between multiple components. For example, a first component may provide a first fluid to a second component and the second component may generate a second fluid from the first fluid. As such, a flow path exists between the first component and the second component.

Biogas may be generated by landfills, organic waste operations, and other means (e.g., wastewater treatment plants, agricultural locations). Accordingly, biogas purification systems may be used to treat a contaminated biogas stream to generate methane-rich product streams (e.g., renewable natural gas). The biogas purification system may include one or more components to facilitate removal of contaminants (e.g., water vapor, nitrogen and oxygen, hydrogen sulfide, non-methane hydrocarbons, inorganic compounds, volatile organic compounds (VOCs), one or more heavy hydrocarbons (e.g., C₆_{⁺})). For example, the one or more components may include a swing adsorption system (e.g., pressure swing adsorption, temperature swing adsorption) and/or a membrane separation system. In general, swing adsorption systems utilize one or more adsorbent beds to enable adsorption and desorption of contaminants. To transition between various stages (e.g., adsorption, desorption), a temperature swing and/or a pressure swing may be used to sequentially operate the adsorbent beds at different temperatures and/or pressures to generated a treated gas. Meanwhile, the membrane separation systems selectively permeate one or more gases (e.g., selectively permeate methane over CO₂ and oxygen (O₂)) from methane from the treated gas to generate one or more product streams (e.g., methane-rich product stream). Put differently, the membrane permeates CO₂ and O₂ such that methane is generated as part of a retentate stream.

Conventional biogas treatment systems use pressure swing adsorption (PSA) technology for the removal undesirable compounds upstream from the membrane separation system as pretreatment. It is desirable to remove contaminants prior to the treated gas contacting the membrane separation system, which may otherwise negatively impact the membranes typically in the form of reduced flux (e.g., capacity), reduce selectivity (e.g., increased methane in waste), or both and it may be desirable to replace the membranes more frequently. Accordingly, a need exists for an improved system capable of removing contaminants to sufficiently low levels to generate methane-rich product streams and extend the lifetime and performance of the membrane separation system.

It is believed that removal of contaminants upstream of the membrane separation systems may generate methane-rich product streams and improve the performance and lifetime of the membrane separation system. Accordingly, this disclosure relates to systems and methods for the use of a TSA system as pretreatment for treating biogas to generate a methane-rich product streams (e.g., gas). In certain embodiments, the TSA system (e.g., adsorbent beds) may consist of one or more adsorbent beds to selectively remove contaminants. For example, the one or more adsorbent beds of the TSA system may operate in parallel and undergo different cycles including adsorption, depressurization, heating, cooling, and repressurization. For example, the TSA system may utilize two or more adsorbent beds, three or more adsorbent beds, four or more adsorbent beds, etc. Accordingly, the embodiments described herein enable treating an incoming gas stream to generate a treated gas stream that is substantially lower in contaminants relative to the incoming gas stream.

In one embodiment, the one or more adsorbent beds may include a media composition. For example, a media composition may include one or more layered materials to enhance the removal of contaminants (e.g., heavy hydrocarbons) relative to existing PSA and other TSA systems. The one or more layered materials may be layered with certain quantities such that the incoming gas stream (e.g., contaminated gas stream, pretreated gas stream, biogas) undergoes adsorption and one or more contaminants are selectively removed (e.g., via adsorption) at each layer. The adsorbent bed may include a first layer (e.g., first material, bottom layer), a second layer (e.g., second material, middle layer), a third layer (e.g., third material, top layer), or more (e.g., a fourth layer, a fifth layer). For example, the first layer may include an activated bed support (e.g., activated alumina, aluminum oxide (Al₂O₃), aluminum oxide containing material) configured to remove a large portion of water from an incoming gas stream and enable flow distribution, the second layer may include Al₂O₃ to remove additional water vapor present in the gas stream, and the top layer may include a molecular sieve (e.g., activated carbon, zeolite, clay) to remove heavy hydrocarbons, VOCs, small quantities of water, and other undesirable components in the gas stream. In one non-limiting example, the fourth layer may include molecular sieves. Accordingly, present embodiments may leverage water-selective materials as part of the first layer and second layer such that sufficient amounts of water are removed from an incoming gas stream. This enables the third layer to be tasked to selectively remove VOCs (e.g., non-methane containing VOCs), hydrocarbons, small quantities of water, and other undesirable contaminants.

Conventional adsorbent beds utilize a ceramic-based media such as Denstone® (available from Saint-Gobain Ceramics $ Plastics, Inc. of Stow, OH) as part of their first layer, which has minimal water adsorption capacity. In contrast, present embodiments utilize an activated bed support as part of the first layer, which advantageously provides additional adsorption capacity of water. In this way, integration of a water-selective material (e.g., activated bed support) in the first layer provides several advantages such as greater moisture removal in a given adsorption step, a decrease in the amount of material in the second layer, fewer regeneration stages, and decreased hydrocarbon losses.

Relatedly, in certain embodiments, the thickness (e.g., quantity, amount) of the one or more layered materials may be modified for selective removal of contaminants. For example, a third of the adsorbent bed may consist of the third layer (e.g., molecular sieve layer) to achieve extremely low levels of contaminants (e.g., removal of heavy hydrocarbons, VOCs, and other contaminants), and to ensure that the adsorbent bed life is sufficient in the event media fouling occurs. It should be noted that while amounts of VOCs may vary depending on the type of landfill, the TSA media described herein may advantageously remove large amounts of VOCs in an untreated gas stream. For example, the TSA media may treat an untreated gas stream that includes about 100 ppm in VOCs to generate a treated gas stream that exhibits about less than or equal to 1 parts per million (ppm) of VOCs in a treated gas stream exiting the TSA system. Accordingly, the media composition of the adsorbent beds provide several advantages, including but not limited to greater moisture removal and selective removal of contaminants (e.g., removal of heavy hydrocarbons, VOCs, and other contaminants).

In one embodiment, a biogas purification system may utilize the disclosed adsorbent bed (e.g., one or more adsorbent beds in parallel) as part of the TSA system to treat an incoming gas stream. For example, the incoming gas stream may first contact the first layer such that the incoming gas stream is treated to generate a first partially treated gas, wherein the first partially treated gas is depleted in water relative to the incoming gas stream. The first partially treated gas may subsequently contact the second layer such that the first partially treated gas stream is treated to generate a second partially treated gas, wherein the second partially treated gas is depleted in water vapor relative to the first partially treated gas. Afterwards, the second partially treated gas may contact the third layer such that the second partially treated gas stream is treated to generate the treated gas, wherein the treated gas is depleted in heavy hydrocarbons, VOCs, and other undesirable components relative to the second partially treated gas. Accordingly, layering the one or more materials in an adsorbent bed enables selective removal of contaminants to generate the treated gas, which may undergo additional treatment by the membrane separation system to generate the methane-rich product stream.

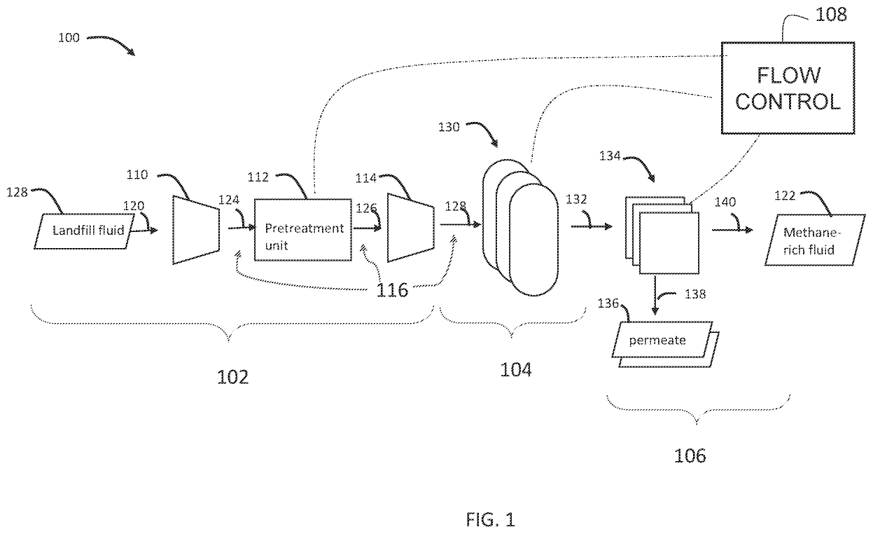

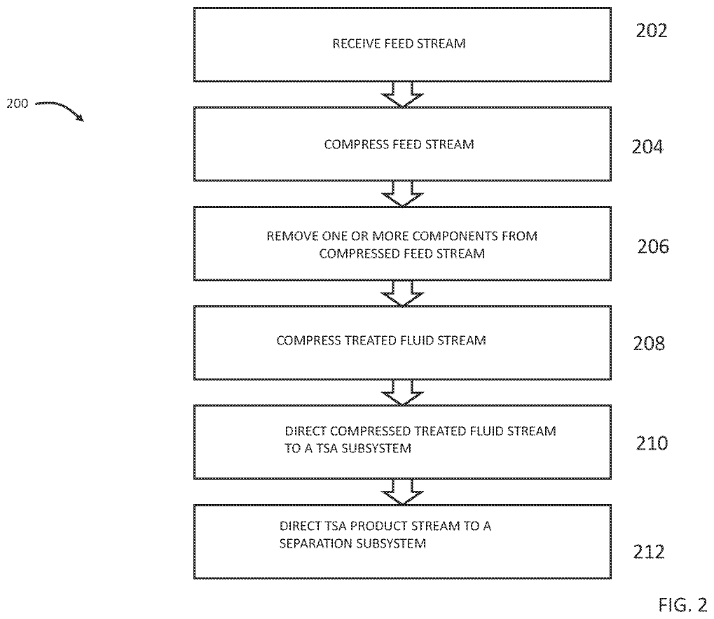

Turning now to the drawings, FIG. 1 illustrates an embodiment of a fluid purification system 100 (e.g., purification system, a biogas purification system) in accordance with the present disclosure. FIG. 2 illustrates a flow diagram of a process 200 related to an embodiment of the fluid purification system 100 of FIG. 1. To facilitate discussion, FIGS. 1 and 2 will be discussed below concurrently. It should be noted that the process 200 is not limiting, and the fluid purification system 100 and/or the process 200 may include additional or fewer steps than those illustrated. Further, the fluid purification system 100 and/or process 200 may include steps that are performed in an alternative order to that illustrated in process 200. That is, certain steps may be performed before, after, or concurrently to/with another respective step.

The fluid purification system 100 may include one or more purification subsystems. The purification subsystems may include a pretreatment subsystem 102, a TSA subsystem 104, a separation subsystem 106, and a flow control subsystem 108. It should be noted that the fluid purification system 100 may include one or more additional subsystems such as a recycle subsystem. Additionally, and/or alternatively, one or more subsystems of the fluid purification system 100 may be omitted. For example, the flow control subsystem 108 may be omitted from the fluid purification system 100. The pretreatment subsystem 102 may include a first compression stage 110, a pretreatment unit 112, a second compression stage 114, or a combination thereof. The pretreatment unit 112 may include a hydrogen sulfide removal unit (e.g., a fixed-bed, a regenerative bed, a lead-lag bed, and the like). The pretreatment unit may also include one or more filters, one or more separators, one or more dehydrators, one or more chillers, and the like.

The TSA subsystem 104 may include one or more regenerative adsorption beds, one or more regenerative adsorption columns, a heating system, a cooling system, one or more switching valves, one or more heat exchangers, or a combination thereof. The separation subsystem 106 may include one or more VOC adsorption beds, one or more membrane separation stages, and the like. The flow control subsystem 108 may include one or more controllers, one or more sensors, one or more manifolds, and the like. The flow control subsystem 108 may control one or more flowrates of one or more produced fluids of the fluid purification system 100. For example, the controllers may provide instructions to the flow control subsystem 108 to vary the flowrates and ratios of produced fluids to the one or more subsystems of the fluid purification system 100 to account for fluid composition, pressure, temperature, operational efficiency, or any combination thereof. In certain embodiments, the controllers may continuously vary the flowrates and ratios of the produced fluids (e.g., gas and/or liquid) to the manifolds in real-time to maintain the desired flow rates within upper and lower thresholds.

In certain embodiments, the flow control subsystem 108 is configured to control operation of the fluid purification system 100, such by controlling modes of operation of the TSA system 130 (e.g., different stages of one or more adsorbent beds, switching between different cycles), controlling operation of the compression processes (e.g., pressure, flow rate) associated with the first compression stage 110 and second compression stage 114, controlling flow rates of one or more streams of the fluid purification system 100, or any combination thereof. In one example, the flow control subsystem 108 may be communicatively coupled with any of the components of the fluid purification system 100. In some embodiments, the flow control subsystem 108 may interface with one or more sensors positioned within the fluid purification system 100. In some embodiments, the flow control subsystem 108 may monitor fluid purification system 100. Accordingly, the flow control subsystem 108 may adjust parameters of the fluid purification system 100 by controlling operation automatically in response to sensor feedback and/or operating parameters, in response to user or operator input or selections, or any combination thereof.

The fluid purification system 100 may include one or more fluid paths 116. The fluid paths 116 may be configured to direct fluids through the purification subsystems of the fluid purification system 100. A landfill fluid 128 may be directed into the fluid purification system 100 as a feed stream 120 for processing (e.g., purification) into a methane-rich fluid 122. The landfill fluid 128 exiting a landfill or entering the fluid purification system 100 may include a methane volume percentage in the range of 30% and 75%, but more typically in the range of 45% and 60%.

At block 202, the compression stage 110 receives the feed stream 120. For example, the feed stream 120 may be directed towards, fed, or otherwise provided, into the first compression stage 110 of the pretreatment subsystem 102. The first compression stage 110 may include one or more coolers (e.g., air coolers, oil coolers) and/or one or more compressors configured to compress the feed stream 120. In some embodiments, the first compression stage 110 may include one or more compressors in parallel. At block 204, the feed stream 120 is compressed, producing a compressed feed stream 124. The compressed feed stream 124 may be compressed to a predetermined pressure. The predetermined pressure may be based on an operational pressure of the pretreatment unit 112. The compressed feed stream 124 may be directed to the pretreatment unit 112. At block 206 of the process 200, the fluid purification system 100 may remove one or more components from the compressed feed stream 124. In some embodiments, the pretreatment unit 112 may be a hydrogen sulfide removal unit. As such, the pretreatment unit 112 may produce a treated fluid stream 126. The treated fluid stream 126 may include a reduced percentage of the one or more components that may include hydrogen sulfide. It should be noted that the implementation of the pretreatment unit 112 described herein may depend on the amount of H₂S that may be present in the feed stream 120. For example, some landfills may have very low H₂S levels, which may permit removal of the pretreatment unit 112. Accordingly, the pretreatment unit 112 (e.g., hydrogen sulfide removal unit) may be optional.

The feed stream 120 includes primarily methane and carbon CO₂, followed by small quantities of contaminants such as water vapor, nitrogen and oxygen (O₂), hydrogen sulfide (H₂S), non-methane hydrocarbons, inorganic compounds, volatile organic compounds (VOCs), heavy hydrocarbons (e.g., C₆_{⁺}). It should be noted that the composition of the feed stream 120 may vary depending on the biogas source (e.g., landfill gas, digester gas). For example, it is presently recognized that digester gas includes greater methane amounts relative to landfill gas. An example composition of a feed stream 120 from the landfill fluid 128 may include about 45% to about 65% of methane, about 45% to about 65% of CO₂, and about 0.001% to about 20% other components and contaminants (e.g., water vapor, O₂, H₂S, non-methane hydrocarbons, inorganic compounds, volatile organic compounds (VOCs), heavy hydrocarbons (e.g., C₆_{⁺})).

In some embodiments, the pretreatment subsystem 102 may remove water by cooling the compressed feed stream 124 to the dewpoint (e.g., about 4° C. and varying with the pressure of the stream) prior to feeding the gas to the TSA system 130. In this way, performing a bulk water removal reduces the amount of water that may otherwise be adsorbed by the TSA system, thereby extending the capacity of the TSA system (i.e., adsorbent beds). The pretreatment unit 112 may generate the treated fluid stream 126 (e.g., partially treated biogas stream), wherein the treated fluid stream 126 may be depleted in contaminants (e.g., water, H₂S) relative to the compressed feed stream 124. The treated fluid stream 126 may be subsequently fed into second compression stage 114, as described below.

At block 208 of the process 200, the fluid purification system 100 may compress the treated fluid stream 126. For example, the treated fluid stream 126 may be compressed by the second compression stage 114. The second compression stage 114 may include one or more coolers (e.g., air coolers, oil coolers) and/or one or more compressors. In some embodiments, the second compression stage 114 may include one or more compressors in parallel. The second compression stage 114 may be controlled via the flow control subsystem 108 to compress the treated fluid stream 126 to a second pressure, generating a compressed treated fluid stream 128. The second pressure may be based on an operational pressure of the TSA subsystem 104. It should be noted, that in some embodiments, pressures of the fluid purification system 100 may be controlled based on design of the fluid purification system 100. That is, the fluid purification system 100 may not include the flow control subsystem 108 and may be designed to operate at a series of set pressures. At block 210 of process 200, the fluid purification system 100 may direct the compressed treated fluid stream 128 to the TSA subsystem 104. In some embodiments, the TSA subsystem 104 may remove undesirable components from the compressed treated fluid stream 128 using one or more regenerative beds 130.

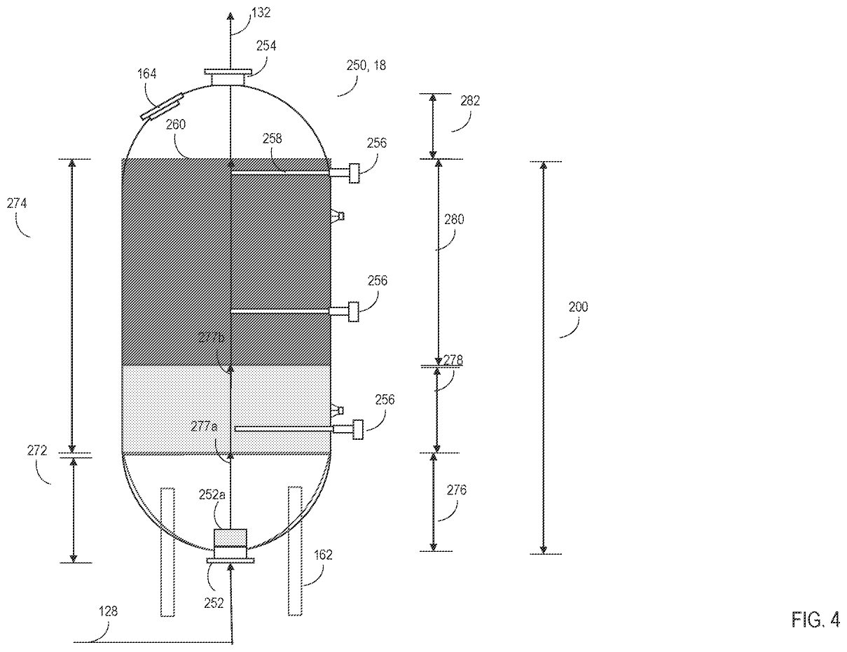

The TSA system 130 may include one or more adsorbent beds (e.g., two or more, three or more, four or more) that operate in parallel and undergo different cycles (e.g., adsorption stage, depressurization stage, heating stage, cooling stage, and repressurization stage) to remove contaminants from the second compressed gas stream 128. For example, TSA system 130 of two adsorbent bed design (e.g., a first adsorbent bed 18a, a second adsorbent bed 18b, as depicted in FIG. 4) may consist of the first adsorbent bed 18a undergoing adsorption (e.g., adsorption stage) to adsorb contaminants from the second compressed gas stream 128. Concomitantly, the second adsorbent bed 18b may cycle through various stages (e.g., depressurization, heating, cooling, and repressurization) to regenerate the second adsorbent bed 18b prior to switching to the adsorption stage.

An exemplary cycle process of the one or more adsorbent beds of the TSA system 130 may include feeding the second compressed gas stream 128 to the first adsorbent bed 130 during an adsorption stage. For example, the second compressed gas stream 128 may exhibit a temperature after cooling at about 4° C. to about 5° C. and pressures ranging from about 100 to about 220 pounds per square inch gauge (psig) (e.g., about 180 psig to about 220 psig). The first adsorbent bed 130 may have been previously regenerated, cooled, and pressurized such that it can adsorb contaminants from the gas stream to produce a gas stream depleted in one or more contaminants, wherein the gas stream depleted in one or more contaminants may be subsequently directed to a polishing bed (e.g., hydrocarbon polishing bed, VOC polishing bed), followed by one or more membrane units. In some embodiments, the gas stream may be optionally directed to one or more additional adsorbent beds (e.g., the second adsorbent bed 130) for additional purification. It should be noted that the gas stream may be depleted in contaminants (e.g., water vapor, non-methane hydrocarbons, inorganic compounds, volatile organic compounds (VOCs), heavy hydrocarbons (e.g., C₆_{⁺})) relative to the second compressed gas stream 128.

After the adsorption stage, the first adsorbent bed 130, may be depressurized (e.g., depressurization stage) to low pressures (e.g., 0-15 psig, about 2 psig, about 4 psig, about 6 psig, about 8 psig, about 10 psig, about 12 psig, or about 14 psig) to facilitate regeneration of the first adsorbent bed 130. For example, depressurization may include a change in pressure from about 100 psig to about 2 psig at about 4° C. to about 5° C. It should be noted that while the first adsorbent bed 130 is undergoing regeneration steps, the second adsorbent bed 130 is removing contaminants from the gas stream generated by the first adsorbent bed 130. Regeneration of the first adsorbent bed 130 begins after the first adsorbent bed 130 reaches an appropriate pressure (e.g., less than or equal to 5 psig). The regeneration stage occurs by thermally heating the first adsorbent bed 130 to temperatures greater than or equal to 140° C. at low pressure (e.g., less than or equal to 5 psig). For example, a heated stream may be provided to the first adsorbent bed 130 to thermally heat the bed and promote desorption of the contaminants that were adsorbed from the second compressed gas stream 128. Accordingly, a waste gas stream may be generated by the first adsorbent bed 130 (or one or more adsorbent beds of the TSA system 130) that includes the contaminants (e.g., water vapor, non-methane hydrocarbons, inorganic compounds, volatile organic compounds (VOCs), heavy hydrocarbons (e.g., C₆_{⁺})).

Subsequently, the first adsorbent bed 130 may be cooled (e.g., cooling stage) for next the adsorption cycle. For example, the first adsorbent bed 130 may be cooled to temperatures about less than or equal to 50° C. at 2 psig. Once the first adsorbent bed 130 has reach an appropriate temperature, the first adsorbent bed 130 may be repressurized (e.g., repressurization stage) to from about 2 psig to pressures associated with the adsorption stage (e.g., 100 to 220 psig), which is the pressure that the first adsorbent bed 130 will maintain once it switches to the adsorption stage. Accordingly, after the first adsorbent bed 130 is repressurized, it will switch to the adsorption stage and repeat the cycles (e.g., adsorption stage, depressurization stage, heating stage, cooling stage, and repressurization stage) such that it can remove contaminants from the second compressed gas stream 128. It should be noted that the cycle processes described herein is with respect to the first adsorbent bed 130 for simplicity. However, any of the one or more adsorbent beds associated with the TSA system 130 undergo similar cycle stages.

After the first adsorbent bed 130 has removed contaminants from the second compressed gas stream 128, the first adsorbent bed 130 may generate a gas stream that has substantially less contaminants from the second compressed gas stream 128. The gas stream may be directed to the second adsorbent bed 130 such that the second adsorbent bed 130 may selectively remove contaminants and generate a treated gas stream 132. The treated gas stream 132 primarily consists of methane, CO₂, nitrogen and O₂ and is significantly depleted in contaminants relative to the second compressed gas stream 128. As such, the TSA subsystem 104 may generate a TSA product stream 132.

At block 212 of the process 200, the fluid purification system 100 may direct the TSA product stream 132 to the separation subsystem 106. In some embodiments, the TSA product stream 132 may be directed to one or more separation units 134. The one or more separation units 134 may include a VOC polishing bed, one or more membrane units, or a combination thereof. The separation subsystem 106 may generate one or more permeate fluids 136 via one or more permeate streams 138. Further, the separation subsystem 106 may generate the methane-rich fluid 122 via a methane-rich stream 140. The methane-rich fluid 122 may be used for one or more applications. The applications may include injecting the methane-rich fluid 122 into a pressure swing adsorption unit (PSA) for nitrogen rejection.

Accordingly, FIG. 1 shows multiple flow paths with the fluid purification system 100. For example, the pretreatment subsystem 102 includes a first flow path formed by the feed stream 120, the compressed feed stream 124, the treated fluid stream 126, and the compressed treated fluid stream 128. Further, the separation subsystem 106 includes a second flow path formed by the TSA product stream and VOC treated stream 132, the permeate stream 138, and the methane-rich stream 140. The TSA subsystem 130 fluidly couples the first flow path to the second flow path.

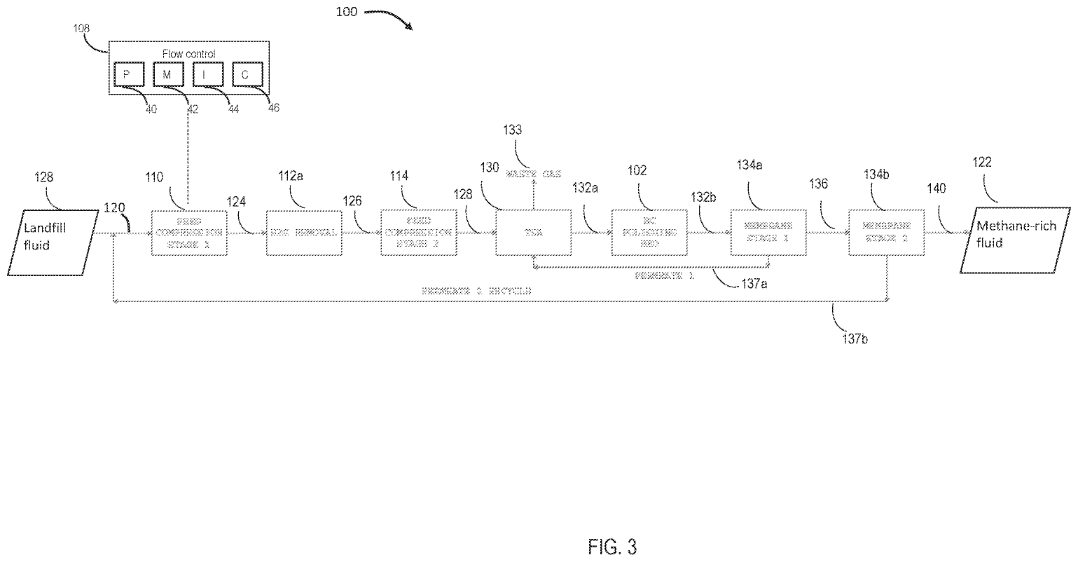

By way of example FIG. 3 is a schematic diagram of an embodiment of the biogas purification system 100 of FIG. 1, in accordance with an aspect of the present disclosure. The fluid purification system 100 may include a first compression stage 110, a pretreatment unit 112 (e.g., H₂S removal unit 112a), a second compression stage 114, a TSA system 130, a hydrocarbon (HC) polishing bed 102, a membrane separation system 134 (e.g., membrane stage I 134a, membrane stage II 134b (i.e., membrane stages)), and a flow control subsystem 108. In a generally similar manner as FIG. 1, the fluid purification system 100 may receive a landfill feed 120 from a raw biogas source (e.g., a landfill fluid 128) and generate a methane-rich product stream 122.

A raw biogas source may provide the landfill feed 120 to the fluid purification system 100 (e.g., the first compression stage 110) via one or more conduits. In general, the landfill feed 120 includes primarily methane and carbon CO₂, followed by small quantities of contaminants such as water vapor, nitrogen and oxygen (O₂), hydrogen sulfide (H₂S), non-methane hydrocarbons, inorganic compounds, volatile organic compounds (VOCs), heavy hydrocarbons (e.g., C₆_{⁺}). The first compression stage 110 may be positioned downstream from the raw biogas source such that it may receive the landfill feed 120 via one or more inlet ports. The landfill feed 120 may be compressed by the first compression stage 110 and subsequently output as compressed feed stream 124. The first compression stage 110 may be in fluid communication with the H₂S removal unit 112 such that the H₂S removal unit 112a may receive the compressed feed stream 124 for pretreating (i.e., removal of H₂S). The H₂S removal unit 112 may generate a treated fluid stream 126, wherein the treated fluid stream 126 may be depleted in contaminants (e.g., H₂S) relative to the compressed feed stream 124. The treated fluid stream 126 may be subsequently fed into the second compression stage 114, wherein the second compression stage 114 may compress the gas to generate a second compressed gas stream 128, which may include a mixture of primarily methane and carbon CO₂, followed by small quantities of contaminants such as water vapor, nitrogen and oxygen (O₂), non-methane hydrocarbons, inorganic compounds, volatile organic compounds (VOCs), heavy hydrocarbons (e.g., C₆_{⁺}). Accordingly, the second compressed gas stream 128 may be directed to the TSA system 130 for additional purification.

In general, the TSA system 130 may include one or more adsorbent beds (e.g., two or more, three or more, four or more) that operate in parallel and undergo different cycles (e.g., adsorption stage, depressurization stage, heating stage, cooling stage, and repressurization stage) to remove contaminants from the second compressed gas stream 128. Accordingly, one or more adsorbent beds of the TSA system 130 may purify the second compressed gas stream 128 to generate a treated gas stream 132 and a waste gas stream 133, as described above. The treated gas stream 132 primarily consists of methane, CO₂, and O₂ and is significantly depleted in contaminants relative to the second compressed gas stream 128. In contrast, the waste gas stream 133 consists primarily of the contaminants that were removed from the second compressed gas stream 128. The waste gas stream 133 is generated as part of regenerating the adsorbent beds (i.e., thermally heating the adsorbent beds via a heated gas stream (e.g., first permeate stream 137a) to facilitate desorption of contaminants).

In some embodiments, the treated gas stream 132 may be directed to the HC polishing bed 102. The HC polishing bed 102 is positioned downstream from the TSA system 130 such that it may act as backup (e.g., optional) to the TSA system 130 if one or more adsorbent beds of the TSA system 130 are nearing the end of their life (i.e., one or more adsorbent beds are inefficient at removing contaminants). In this way, the HC polishing bed 102 acts as an “oil catch” to remove volatile organic compounds (VOCs) and/or heavy hydrocarbons (e.g., C₆_{⁺}), thereby mitigating downstream damage that would otherwise occur to the membrane separation system 134 (e.g., first membrane stage 134a, second membrane stage 134b). For example, the HC polishing bed 102 may consist of a non-regenerable bed to remove residual contaminants from the incoming treated gas stream 132 and subsequently generate a second treated gas stream 132a. The second treated gas stream 132a may be depleted in contaminants (e.g., volatile organic compounds (VOCs) and/or heavy hydrocarbons (e.g., C₆_{⁺})) relative to the treated gas stream 132. The fluid purification system 100 may subsequently direct the second treated gas stream 132a to the membrane separation system (e.g., first membrane stage 134a, second membrane stage 134b) for additional processing.

As indicated in FIG. 1, the membrane separation system 134 includes one or more membranes (e.g., first membrane stage 134a, second membrane stage 134b) that are configured to selectively permeate CO₂ and O₂ over methane from an incoming stream. In the illustrated example, first membrane stage 134a is positioned downstream from the HC polishing bed 102 such that it may receive the second treated gas stream 132a, which primarily consists of a mixture including methane, nitrogen, CO₂, and O₂. The first membrane stage 134a may separate the second treated gas stream 132a by selectively permeating CO₂ and O₂ and generate two or more streams, wherein the two or more streams include the first permeate stream 137a and a partially enriched methane stream 136. The partially enriched methane stream 136 is enriched in methane and may include small quantities of CO₂ and nitrogen and O₂. Accordingly, the partially enriched methane stream 136 may be directed to the second membrane stage 134b for additional processing (e.g., separation of remaining quantities of CO₂ and O₂).

In some embodiments, the first permeate stream 137a may be directed back to the TSA system 130 for downstream uses. The first permeate stream 137a is enriched in CO₂ and O₂ (e.g., about 95% CO₂) and may include small quantities of methane relative to the second treated gas stream 132a. The first permeate stream 137a may be provided to one or more adsorbent beds during a regeneration stage and utilized as a heat source (e.g., heated stream) to thermally heat the one or more adsorbent beds, thereby promoting desorption of the contaminants that were adsorbed from the second compressed gas stream 128. It should be noted, in some embodiments, the fluid purification system 100 may direct the first permeate stream 137a to a thermal oxidizer for destruction.

The second membrane stage 134b is positioned downstream from the first membrane stage 134a such that it may receive the partially enriched methane stream 136, which consists primarily of methane and small quantities of CO₂ and O₂ relative to the second treated gas stream 132a. The second membrane stage 134b may separate the partially enriched methane stream 136 by selectively permeating residual CO₂ and O₂ and generate two or more streams, wherein the two or more streams include the methane-rich product stream 122 and a second permeate recycle stream 137b. The second permeate recycle stream 137b may be recycled back via a conduit and combined with a stream including the landfill feed 120, which may be subsequently provided to the first compression stage 110. Accordingly, it should be noted that in some embodiments, the landfill feed 120 may include a mixture including the landfill feed 120 obtained from a raw biogas source and the second permeate recycle stream 137b. Furthermore, while the illustrated diagram illustrates two membrane stages as part of the membrane separation system 134, additional membrane stages may be utilized to obtain high quality, methane-rich product stream 122. It should be noted, that a portion of the above-mentioned flow paths may be directed to a thermal oxidizer at one or more points within the fluid purification system 100. For example, a portion of the first flow path formed by the feed stream 120, the compressed feed stream 124, the treated fluid stream 126, and the second compressed gas stream 128 may be directed to the thermal oxidizer. The thermal oxidizer may include an elevated stack and may be used to destroy the portion of the first flow path through combustion.

In a generally similar manner as FIG. 1, the flow control subsystem 108 is configured to control all aspects of the fluid purification system 100. For example, the flow control subsystem 108 is configured to receive sensor feedback from one or more sensors coupled to the fluid purification system 100 (e.g., the first compression stage 110, the H₂S removal unit 112a, the second compression stage 114, the TSA system 130, the HC polishing bed 102, the membrane separation system 134 (e.g., first membrane stage 134a, second membrane stage 134b)) and/or additional components of the fluid purification system 100 and control the same equipment based on the sensor feedback, operating modes, user input, computer models, or any combination thereof. While not illustrated, the sensors may include temperature sensors, pressure sensors, flow rate sensors, gas composition sensors, or any combination thereof. Additionally and/or alternatively, one or more subsystems of the fluid purification system 100 may be omitted. For example, the flow control subsystem 108 may be omitted from the fluid purification system 100. It should be noted, that in some embodiments, pressures of the fluid purification system 100 may be controlled based on design of the fluid purification system 100. That is, the fluid purification system 100 may not include the flow control subsystem 108 and may be designed to operate at a series of set pressures.

In certain embodiments, the flow control subsystem 108 is configured to control operation of the fluid purification system 100, such by controlling modes of operation of the TSA system 130 (e.g., different stages of one or more adsorbent beds, switching between different cycles), controlling operation of the compression processes (e.g., pressure, flow rate) associated with the first compression stage 110 and second compression stage 114, controlling flow rates of one or more streams of the fluid purification system 100, or any combination thereof. In one example, the flow control subsystem 108 may be communicatively coupled with any of the components of the fluid purification system 100. In some embodiments, the flow control subsystem 108 may interface with one or more sensors positioned within the fluid purification system 100. In some embodiments, the flow control subsystem 108 may monitor system 100. Accordingly, the flow control subsystem 108 may adjust parameters of the fluid purification system 100 by controlling operation automatically in response to sensor feedback and/or operating parameters, in response to user or operator input or selections, or any combination thereof.

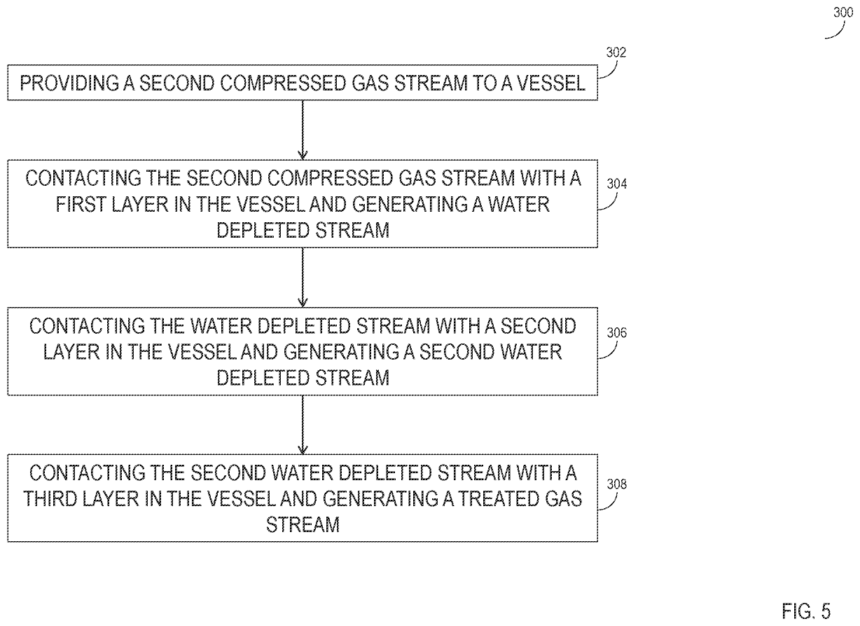

With the foregoing in mind, FIG. 4 is a schematic diagram of an adsorbent bed of a temperature swing adsorption (TSA) system 130 of FIGS. 1 and 2, in accordance with an aspect of the present disclosure. FIG. 5 is a flow diagram related to the embodiment of the TSA adsorbent bed of FIG. 4. To facilitate discussion, FIGS. 4 and 5 will be discussed below concurrently. It should be noted that the process 300 is not limiting, and the TSA adsorbent bed and/or the process 300 may include additional or fewer steps than those illustrated. Further, the TSA adsorbent bed and/or process 300 may include steps that are performed in an alternative order to that illustrated in process 300. That is, certain steps may be performed before, after, or concurrently to or with another respective step.

In general, it should be noted that TSA system 130, adsorbent beds 18a, 18b, and TSA vessel 250, TSA vessel system 250, (e.g., vessel 250, vessel system 250) may be used interchangeably. In the illustrated embodiment, a TSA vessel 250 includes an inlet 252, a bottom distribution system 252a (e.g., a v-wire screen), an outlet 254, a thermal well, temperature probe, or transmitter 256, a level 258, a weld seam 260, vertical legs 162, and a manway 164. It should be noted that a v-wire screen (e.g., similar to bottom distribution system 252a) may be installed as part of the outlet 254. Accordingly, the TSA vessel 250 may receive the second compressed gas stream 128 via the inlet 252 and generate the treated gas stream 132.

In some embodiments, the TSA vessel 250 may be stabilized by the vertical legs 162, which act as support structures. While the illustrated embodiment shows two vertical legs 162 (e.g., extensions, panels, supports), any suitable support structure and/or number of support structures may be used to support the vessel 250. The TSA vessel may include a bottom portion 272 and a top portion 274. The bottom portion 272 may include one or more media disposed in the bottom, wherein the one or more media may include a first layer 276 (e.g., including a first material). The top portion 274 may include two or more media layers, wherein the two or more media layers include a second layer 278 (e.g., including a second material) and the third layer 280 (e.g., including a third material). The second layer 278 is downstream of the first layer 276 with respect to the flow of the second compressed gas stream 128 through the inlet 252 (e.g., a fluid flow flowing from the inlet 252 to the outlet 254). The third layer 280 is downstream on the second layer 278 with respect to the flow of the second compressed gas stream 128 through the inlet 252. In the illustrated diagram, one or more levels 258 may be distributed throughout the vessel 250 to help level the layered materials. For example, the levels 258 may be a long paddle to facilitate keeping the materials substantially flat or level within the vessel 250.

In some embodiments, each layer (e.g., first layer 276, second layer 278, third layer 280) of the vessel 250 includes a particular material that is capable of removing one or more contaminants from the second compressed gas stream 128 as it flows through the vessel. For example, the first material of the first layer 276 may include an activated bed support containing aluminum oxide containing material that removes water. The second material of the second layer 278 may include aluminum oxide containing material that also removes water but relatively less than the first layer 276. The third material of the third layer 280 may include a molecular sieve (e.g., activated carbon, zeolites, clay). In this way, as the second compressed gas stream 128 flows through the vessel 250, one or more contaminants is selectively removed at each layered to produce a more purified gas stream relative to the second compressed gas stream 128.

As described above, the first layer 276, the second layer 278, and the third layer 280 may include different materials. Further, the volume and/or height of the first layer 276, the second layer 278, and the third layer 280 may be different. In one example, the first layer 276 may be filled up to the tangent line of the vessel (e.g., lower head), which may cover the v-wire screen (e.g., bottom distribution unit 252a). As one non-limiting example, the TSA media 200 on a weight basis may include a first layer 276 that is about 1-20 wt % of the total weight of the TSA media 200, 20-40% of the second layer 278, and the remaining wt % (e.g., 40-about 80 wt %) may be the third layer 280. For example, the vessel 250 may include a larger volume of the third layer 280 as compared to the second layer 278. For example, a volume ratio of the third layer 280 to the second layer 278 may be range between about ¼, ⅔, ¾, 5/4, 3/2, 2, 7/3, or 3. In some embodiments, the volume of the third layer 280 may be greater than the combined volume of the second layer 278 and the first layer 276. For example, a volume ratio of the third layer 280 to the combined second layer 278 and the first layer 276 may be range between about ¼ to about 5, such as between about ¼ to about 3, about ¼ to about 2, about ¼ to about 1, about ¼, ⅔, ¾, ⅞, 1, 5/4, 3/2, or 2. It should be noted that while the illustrated diagram depicts, three layers, in some embodiments, an optional fourth layer may be provided to a portion of the TSA vessel 250. For example, the fourth layer may include a molecular sieve and may be layered upstream the first layer 276 or downstream the third layer 280. In another example, a TSA vessel 250 may include a full bed support (e.g., activated bed support), which may enable use of two layers (e.g., second layer 278, third layer 280). For example, the two layers may include a first layer selective towards water (e.g., water-adsorbing material, such as aluminum oxide containing material, second layer 278) and a second layer 278 selective towards hydrocarbons, VOCs, and other undesirable contaminants (e.g., a hydrocarbon/VOCs/etc.—selective layer, such as molecular sieves, third layer 280). (e.g., third layer 280) by volume. For example, the second layer 278 may be above the top weld seam of the TSA vessel 250 in the two-layer configuration. Accordingly, it should be noted that relative ordering of the layers may be modified based on the composition of the incoming gas stream.

In some embodiments, the TSA vessel 250 may be layered by providing a first material through the manway 164 (i.e., provides access to inside of the vessel 250) to generate the first layer 276. For example, the first material may be packed along a first length associated with the first layer 276. In some embodiments, the bottom distribution system 252a, which consists of a v-wire basket, may be present at the bottom portion 272 of the vessel 250 to enable distribution of the first material (e.g., first layer 276). It should be noted that the composition of the first layer 276 includes a material that is selective towards one or more contaminants (e.g., water, water vapor). Accordingly, the first layer 276 may include an activated bed support (ABS) and may include aluminum oxide containing material to adsorb water from the second compressed gas stream 128.

In some embodiments, following the addition of the first material, a second material may be provided to the vessel 250 through the manway 164, wherein the second material may be packed along a second length associated with the second layer 278, thereby generating the second layer 278. It should be noted that the composition of the second layer 278 may be selected such that the material is selective towards one or more contaminants (e.g., water, water vapor). Accordingly, the second layer 278 may include aluminum oxide containing material to facilitate adsorption of water vapor.

In some embodiments, following the addition of the second material, a third material may be provided to the vessel 250 through the manway 164, wherein the third material may be packed along a third length associated with the third layer 280 to generate the third layer 280. It should be noted that the composition of the third layer 280 may be selected such that the material is selective towards one or more contaminants (e.g., VOCs, heavy hydrocarbons (e.g., C₆_{⁺})). Accordingly, the third layer 280 may include a molecular sieve material (e.g., activated carbon, zeolites, clays) to adsorb VOCs, heavy hydrocarbons, or a combination thereof. The vessel 250 and/or third layer 280 may be filled to the tangent of the head, which may be above the weld seam 260, as indicated in the diagram, such that the top-most portion of the vessel 250 may include an empty space (e.g., region 282) to facilitate flow of a stream. Accordingly, the combination of the first material in the first layer 276, the second material in the second layer 278, and the third material in the third layer 280 may be collectively referred to as the TSA media 200.

In some embodiments, the vessel 250 may also include one or more thermal well, temperature probes, or transmitters 256 distributed throughout the vessel 250 to obtain sensor feedback (e.g., temperature readings, pressure readings, chemical composition readings) associated with the vessel 250. For example, the thermal well, temperature probe, or transmitter 256 may acquire sensor feedback as the vessel 250 of the various cycles as described in FIGS. 1 and 3, which enables determining when to cycle to a subsequent stage as part of the TSA system 130.

It should be noted that the material associated with each layer may provide several advantages. As indicated above, certain conventional adsorbent beds may utilize a ceramic-based media such as Denstone® as part of their first layer, which a minimal water adsorption capacity. In contrast, present embodiments utilize an activated bed support as part of the first layer 276, which selectively removes water from a gas stream (e.g., second compress gas stream 32). In this way, integration of a water-selective material (e.g., activated bed support) in the first layer provides several advantages such as additional adsorption capacity of water in a given adsorption step (i.e., greater removal of water), a decrease in the amount of material in the second layer, fewer regeneration stages of the vessel 250, and decreased hydrocarbon losses. In a generally similar regard, the composition of the second layer 278 is selective towards adsorption of water vapor. This enables the full adsorption capacity of the third layer 280 (e.g., molecular sieve layer) to be utilized for removing VOCs, heavy hydrocarbons, and other undesirable contaminants.

In some embodiments, the thickness of the one or more layered materials may be modified for selective removal of contaminants. In the illustrated diagram, a significant percentage of the top portion 274 consists of the third layer 280. For example, about 70% of the top portion 274 may consist of the third layer 280 (e.g., material associated with the third layer 280), while about 30% of the top portion 274 may consist of the second layer 278 (e.g., material associated with the second layer 278). It is presently recognized that filling the vessel 250 and/or the top portion 274 primarily with the third layer 280 enables efficient removal of heavy hydrocarbons, VOCs, and other undesirable contaminants from the second water depleted stream 277b to low ppm amounts of contaminants. For example, low ppm amounts may include less than or equal to 10 ppm of contaminants. It should be noted that lower ppm amounts (e.g., less than or equal to 1 ppm) may be achievable if a fresh TSA media bed is utilized. As such, the TSA media blend (e.g., a combination of the first material of the first layer 276, the second material of the second layer 278, and third material of the third layer 280) enhances removal of heavy hydrocarbons over traditional PSA and conventional TSA systems. Furthermore, oversizing the third layer 280 within the top portion 274 or the vessel 250 (e.g., adsorbent bed) ensures that the bed life is sufficient if media fouling occurs. Accordingly, the relative percentage of the third layer 280 may generally be greater than the percentage of the second layer 278.

Accordingly, the composition of the TSA media 200 within the vessel 250 provides several advantages. For example, the overall composition of the media within each layer (e.g., first layer 276, second layer 278, third layer 280) operate synergistically to adsorb contaminants from the second compressed gas stream to sufficiently low amounts 128, thereby generating a treated gas stream 270 that is sufficiently low in water, VOCs, hydrocarbons, and other undesirable components. This provides several advantages downstream, as the treated gas stream 270 will be enriched in methane, CO₂, and O₂ and depleted in contaminants that would otherwise be harmful to downstream components (e.g., membranes of the membrane separation system 134). In this way, the composition of the TSA media 200 of the vessel 250 described herein reduces the frequency at which membranes may need to be replaced, thereby ensuring a longer membrane life.

At block 302, the second compressed gas stream 128 may be provided to the vessel 250. For example, the second compressed gas stream 128, which may include a mixture of primarily methane and CO₂, followed by small quantities of contaminants such as water vapor, nitrogen and oxygen (O₂), non-methane hydrocarbons, inorganic compounds, volatile organic compounds (VOCs), heavy hydrocarbons (e.g., C₆_{⁺}), may be provided to the vessel 250 during the adsorption stage.

At block 304, the second compressed gas stream 128 may contact the first layer 276 in the vessel 250 and generate a first water depleted stream 277a. For example, the first material of the first layer 276 may adsorb water from the second compressed gas stream 128 to generate the first water depleted stream 277a upstream from the first layer 276. In general, the first water depleted stream 277a is depleted in water (e.g., water vapor) relative to the second compressed gas stream 128. The first water depleted stream 277a may continue to flow (e.g., flow upwards, flow downwards) within the vessel 250.

At block 306, the first water depleted stream 277a may contact the second layer 278 in the vessel 250 and generate a second water depleted stream 277b. For example, the second material of the second layer 278 may adsorb additional water (e.g., water vapor) from the first water depleted stream 277a to generate the second water depleted stream 277b. In general, the second water depleted stream 277b is depleted in water (e.g., water vapor) relative to the first water depleted stream 277a. The second water depleted stream 277b may continue to flow upwards within the vessel 250.

At block 308, the second water depleted stream 277b may contact the third layer 280 in the vessel 250 and generate a treated gas stream 132. For example, the third material of the third layer 180 may adsorb of heavy hydrocarbons, VOCs, and other undesirable contaminants to generate the treated gas stream 132 (e.g., treated gas stream 132, second treated gas stream 132a of FIGS. 1 and 3). Accordingly, the treated gas stream 132 may be depleted in of heavy hydrocarbons, VOCs, and other undesirable contaminants relative to the second water depleted stream 277b. While the illustrated diagram only depicts one vessel 250, it should be noted that the treated gas stream 132 may be directed to a VOC polishing bed for removal of VOCs. Afterwards, the treated gas stream 132 may be directed to the membrane separation system 134 for additional processing (i.e., separation of CO₂ and O₂ from the treated gas stream 132 to generate methane-rich product stream 122). It should be noted that in some embodiments, the first water depleted stream 277a and second water depleted stream 277b may be referred to as a treated gas stream.

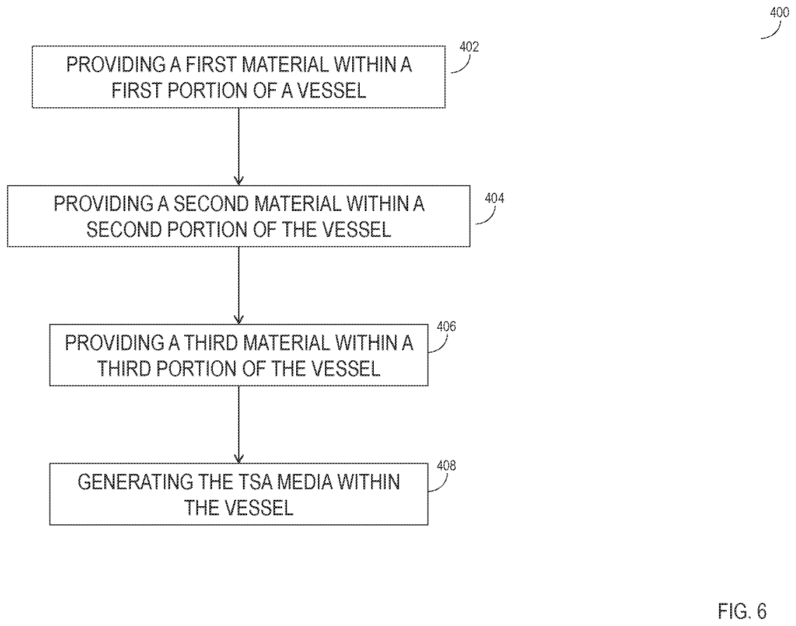

FIG. 6 is a flow diagram related to the embodiment of the TSA adsorbent bed (e.g., TSA vessel 250) of FIG. 3, in accordance with an aspect of the present disclosure. It should be noted that the blocks of process 400 may be performed in the order disclosed herein or in any suitable order. For example, certain blocks of the process may be performed concurrently. In addition, in certain embodiments, at least one of the blocks of the process 400 may be omitted.

At block 402 of the process 400, a first material may be provided within a first portion to the vessel 250. The first material may be packed along a first length associated with the first layer 276. For example, the first material may consist of a material, such as ABS (e.g., activated alumina, aluminum oxide containing material) that will selectively adsorb one or more contaminants (e.g., water, water vapor).

At block 404 of the process 400, a second material may be provided within a second portion to the vessel 250. The second material may be packed along a second length associated with the second layer 278. For example, the second material may consist of a material, such as aluminum oxide that will selectively adsorb one or more contaminants (e.g., water, water vapor).

At block 406 of the process 400, a third material may be provided within a third portion to the vessel 250. The third material may be packed along a first length associated with the third layer 280. For example, the third material may consist of a material, such as molecular sieves that will selectively adsorb one or more contaminants (e.g., VOCs, heavy hydrocarbons, other undesirable contaminants). At block 408, the TSA media 200 is generating within the vessel, wherein the TSA media 200 includes a combination of the first material in the first layer 276, the second material in the second layer 278, and the third material in the third layer 280.

Technical effects of the disclosed embodiments include a biogas purification system 10, 100 for utilizing a TSA media 200 within a vessel 250 (e.g., adsorbent bed) of a TSA system 130 for removing contaminants within a stream (e.g., landfill feed 120, second compressed gas stream 128) to generated a treated gas stream 132 (e.g., treated gas stream 132, second treated gas stream 132a of FIGS. 1 and 2). The vessel 250 includes the TSA media 200, which is a combination of a first layer 276, a second layer 278, and a third layer 280, wherein each layer includes a material (e.g., a first material, a second material, a third material) that selectively adsorbs one or more contaminants from the second compressed gas stream 128. Advantageously, by generating the TSA media 200, significant amounts of contaminants may be removed during an adsorption stage of the vessel 250, thereby reducing the number of contaminants that would otherwise negatively impact downstream processes. The disclosed techniques may result in improved efficiency of downstream components such as the membrane separation system 134 by removing significant amounts of contaminants during the adsorption stage of the TSA system 130, thereby generating high quality methane-rich product stream 122.

While only certain features and embodiments have been illustrated and described, many modifications and changes may occur to those skilled in the art, such as variations in sizes, dimensions, structures, shapes and proportions of the various elements, values of parameters, such as temperatures and pressures, mounting arrangements, use of materials, colors, orientations, and so forth, without materially departing from the novel teachings and advantages of the subject matter recited in the claims. The order or sequence of any process or method steps may be varied or re-sequenced according to alternative embodiments. It is, therefore, to be understood that the appended claims are intended to cover all such modifications and changes as fall within the true spirit of the disclosure.

Having described various systems, methods, and processes, certain embodiments can include, but are not limited to:

In a first aspect, a system comprises: a temperature swing adsorption (TSA) vessel comprising: an inlet and an outlet configured to provide a fluid flow through the TSA vessel; and a TSA media disposed within the TSA vessel, wherein the TSA media comprises: a first layer comprising a first material; a second layer comprising a second material configured to remove water from the fluid flow, wherein the second layer is downstream of the first layer with respect to the fluid flow; and a third layer comprising a third material configured to remove one or more hydrocarbons, volatile organic compounds (VOCs), or a combination thereof, from the fluid flow, wherein the third layer is downstream of the second layer with respect to the fluid flow, wherein the TSA media is configured to generate a treated gas stream based on the fluid flow, and wherein a volume of the third layer is greater than a volume of the second layer.

A second aspect can include the system of the first aspect, wherein the first material comprises aluminum oxide containing material.

A third aspect can include the system of the first or second aspect, wherein the first layer occupies about 30% by volume of the TSA vessel.

A fourth aspect can include the system of any one of the proceeding aspects, wherein the second material comprises a molecular sieve material.

A fifth aspect can include the system of any one of the proceeding aspects, comprising one or more compression stages configured to generate a compressed gas stream based on a methane-containing feed stream; and wherein the inlet of the TSA vessel is configured to receive the compressed gas stream.

A sixth aspect can include the system of any one of the proceeding aspects, comprising one or more membrane stages configured to receive the treated gas stream and generate a methane-rich fluid based on the treated gas stream.

A seventh aspect can include the system of any one of the proceeding aspects, wherein the volume of the third layer is greater than a sum of the volume of the second layer and a volume of the first layer.

An eighth aspect can include the system of any one of the proceeding aspects, wherein a ratio of the volume of the third layer to the volume of the second layer is greater than about 2.

A ninth aspect can include the system of any one of the proceeding aspects, wherein the third layer is below a weld seam of the TSA vessel.

In a tenth aspect, a method, comprises: providing a compressed gas stream flow to a TSA vessel; contacting the compressed gas stream flow with a first layer of first material within the TSA vessel to generate a first water depleted stream; contacting the first water depleted stream with a second layer of a second material within the TSA vessel to generate a second water depleted stream; and contacting the second water depleted stream with a third layer of a third material within the TSA vessel to generate a treated gas stream.

An eleventh aspect can include the method of the tenth aspect, wherein contacting the first water depleted stream with the second material comprises removing water from the compressed gas stream flow.

A twelfth aspect can include the method of the tenth or eleventh aspect, wherein contacting the second water depleted stream with the third material comprises removing one or more hydrocarbons, VOCs, or a combination thereof, from the compressed gas stream flow.

A thirteenth aspect can include the method of any one of the tenth to twelfth aspects, further comprising: receiving a methane-containing feed gas flow; compressing the methane-containing feed gas flow; and cooling the methane-containing feed gas flow below the dew-point of water to generate the compressed gas stream flow.

A fourteenth aspect can include the method of any one of the tenth to thirteenth aspects, further comprising: providing the treated gas stream to one or more membrane stages disposed outside of the TSA vessel to generate a methane-rich stream.

In a fifteenth aspect, a method, comprises: providing a first material within a first portion of a vessel; providing a second material within a second portion of the vessel; and

providing a third material within a third portion of the vessel, thereby generating TSA media within the vessel, and wherein a volume of the third portion is greater than a volume of the second portion.

A sixteenth aspect can include the method of the fifteenth aspect, wherein the first material and the second material comprise an alumina-containing material.

A seventeenth aspect can include the method of the fifteenth or sixteenth aspect, wherein a volume of the third portion is greater than a sum of the volume of the second portion and a volume of the first portion.

An eighteenth aspect can include the method of any one of the fifteenth to seventeenth aspects, wherein the third material comprises a molecular sieve material.

A nineteenth aspect can include the method of any one of the fifteenth to eighteenth aspects, wherein providing the third material within the third portion of the vessel comprises filling the vessel above a weld seam of the vessel.

A twentieth aspect can include the of any one of the fifteenth to nineteenth aspects, wherein a ratio of the volume of the third portion to the volume of the second portion is greater than about 2.

In a twenty first aspect, a system comprises: a temperature swing adsorption (TSA) vessel comprising: an inlet and an outlet configured to provide a fluid flow through the TSA vessel; and a TSA media disposed within the TSA vessel, wherein the TSA media comprises: a first layer comprising a first material configured to remove water from the fluid flow, wherein the first layer is downstream with respect to the fluid flow; and a second layer comprising a second material configured to remove one or more hydrocarbons, volatile organic compounds (VOCs), or a combination thereof, from the fluid flow, wherein the second layer is downstream of the first layer with respect to the fluid flow, wherein the TSA media is configured to generate a treated gas stream based on the fluid flow, and wherein a volume of the second layer is greater than a volume of the first layer.

While preferred embodiments have been shown and described, modifications thereof can be made by one skilled in the art without departing from the scope or teachings herein. The embodiments described herein are exemplary only and are not limiting. Many variations and modifications of the systems, apparatus, and processes described herein are possible and are within the scope of the disclosure. For example, the relative dimensions of various parts, the materials from which the various parts are made, and other parameters can be varied. Accordingly, the scope of protection is not limited to the embodiments described herein, but is only limited by the claims that follow, the scope of which shall include all equivalents of the subject matter of the claims. Unless expressly stated otherwise, the steps in a method claim may be performed in any order. The recitation of identifiers such as (a), (b), (c) or (1), (2), (3) before steps in a method claim are not intended to and do not specify a particular order to the steps, but rather are used to simplify subsequent reference to such steps.

Also, techniques, systems, subsystems, and methods described and illustrated in the various implementations as discrete or separate may be combined or integrated with other systems, modules, techniques, or methods without departing from the scope of the present disclosure. Other items shown or discussed as directly coupled or communicating with each other may be indirectly coupled or communicating through some interface, device, or intermediate component, whether electrically, mechanically, or otherwise. Other examples of changes, substitutions, and alterations are ascertainable by one skilled in the art and could be made without departing from the spirit and scope disclosed herein.

Each and every claim is incorporated into the specification as an aspect of the present disclosure. Thus, the claims are a further description and are an addition to the aspects of the present disclosure. The discussion of a reference herein is not an admission that it is prior art to the presently disclosed subject matter, especially any reference that may have a publication date after the priority date of this application. The disclosures of all patents, patent applications, and publications cited herein are hereby incorporated by reference, to the extent that they provide exemplary, procedural or other details supplementary to those set forth herein. In the event of conflict, the present specification, including definitions, is intended to control.

What is claimed is:

1. A system comprising:
a temperature swing adsorption (TSA) vessel comprising:
an inlet and an outlet configured to provide a fluid flow through the TSA vessel; and
a TSA media disposed within the TSA vessel, wherein the TSA media comprises:
a first layer comprising a first material;
a second layer comprising a second material configured to remove water from the fluid flow, wherein the second layer is downstream of the first layer with respect to the fluid flow; and
a third layer comprising a third material configured to remove one or more hydrocarbons, volatile organic compounds (VOCs), or a combination thereof, from the fluid flow, wherein the third layer is downstream of the second layer with respect to the fluid flow, wherein the TSA media is configured to generate a treated gas stream based on the fluid flow, and wherein a volume of the third layer is greater than a volume of the second layer.

2. The system of claim 1, wherein the first material comprises aluminum oxide containing material.

3. The system of claim 1, wherein the first layer occupies about 30% by volume of the TSA vessel.

4. The system of claim 1, wherein the second material comprises a molecular sieve material.

5. The system of claim 1, comprising one or more compression stages configured to generate a compressed gas stream based on a methane-containing feed stream; and wherein the inlet of the TSA vessel is configured to receive the compressed gas stream.

6. The system of claim 1, comprising one or more membrane stages configured to receive the treated gas stream and generate a methane-rich fluid based on the treated gas stream.

7. The system of claim 1, wherein the volume of the third layer is greater than a sum of the volume of the second layer and a volume of the first layer.

8. The system of claim 1, wherein a ratio of the volume of the third layer to the volume of the second layer is greater than about 2.

9. The system of claim 1, wherein the third layer is below a weld seam of the TSA vessel.

10. A method, comprising:
providing a compressed gas stream flow to a TSA vessel;
contacting the compressed gas stream flow with a first layer of first material within the TSA vessel to generate a first water depleted stream;
contacting the first water depleted stream with a second layer of a second material within the TSA vessel to generate a second water depleted stream; and
contacting the second water depleted stream with a third layer of a third material within the TSA vessel to generate a treated gas stream.

11. The method of claim 10, wherein contacting the first water depleted stream with the second material comprises removing water from the compressed gas stream flow.

12. The method of claim 10, wherein contacting the second water depleted stream with the third material comprises removing one or more hydrocarbons, VOCs, or a combination thereof, from the compressed gas stream flow.

13. The method of claim 10, further comprising:
receiving a methane-containing feed gas flow;
compressing the methane-containing feed gas flow; and
cooling the methane-containing feed gas flow below the dew-point of water to generate the compressed gas stream flow.

14. The method of claim 10, further comprising:
providing the treated gas stream to one or more membrane stages disposed outside of the TSA vessel to generate a methane-rich stream.

15. A method, comprising:
providing a first material within a first portion of a vessel;
providing a second material within a second portion of the vessel; and
providing a third material within a third portion of the vessel, thereby generating TSA media within the vessel, and wherein a volume of the third portion is greater than a volume of the second portion.

16. The method of claim 15, wherein the first material and the second material comprise an alumina-containing material.

17. The method of claim 15, wherein a volume of the third portion is greater than a sum of the volume of the second portion and a volume of the first portion.

18. The method of claim 15, wherein the third material comprises a molecular sieve material.

19. The method of claim 15, wherein providing the third material within the third portion of the vessel comprises filling the vessel above a weld seam of the vessel.

20. The method of claim 15, wherein a ratio of the volume of the third portion to the volume of the second portion is greater than about 2.

21. A system comprising:
a temperature swing adsorption (TSA) vessel comprising:
an inlet and an outlet configured to provide a fluid flow through the TSA vessel; and
a TSA media disposed within the TSA vessel, wherein the TSA media comprises:
a first layer comprising a first material configured to remove water from the fluid flow, wherein the first layer is downstream with respect to the fluid flow; and
a second layer comprising a second material configured to remove one or more hydrocarbons, volatile organic compounds (VOCs), or a combination thereof, from the fluid flow, wherein the second layer is downstream of the first layer with respect to the fluid flow, wherein the TSA media is configured to generate a treated gas stream based on the fluid flow, and wherein a volume of the second layer is greater than a volume of the first layer.
